(19) United States
(12) Patent Application Publication
(10) Pub. No.: US 2024/0000979 A1
(43) Pub. Date: Jan. 4, 2024

(54) METHODS AND COMPOSITIONS FOR SYNERGISTIC UPTAKE AND RETENTION OF SMALL MOLECULE LIGANDS

(71) Applicant: CORNELL UNIVERSITY, Ithaca, NY (US)

(72) Inventor: Neil H. BANDER, New York, NY (US)

(21) Appl. No.: 18/247,427

(22) PCT Filed: Sep. 28, 2021
(86) PCT No.: PCT/US2021/052271

§ 371 Date: Mar. 30, 2023

Related U.S. Application Data
(60) Provisional application No. 63/086,216, filed on Oct. 1, 2020.

(51) Int. Cl. A61K 51/10

(52) U.S. Cl. CPC A61K 51/1072

(57) ABSTRACT

The present application relates to methods of treating a subject for cancer. The method involves providing a first agent comprising a first targeting component coupled to a first cancer therapeutic component and providing a second agent comprising a second targeting component coupled to a second cancer therapeutic component. The first and second targeting components have different biodistributions and/or pharmacokinetics in the subject. The first and second agents are administered no more than eight hours apart from each other, to the subject to treat cancer. As a result of the administering, the amount of first and second cancer therapeutic component internalized and retained within a tumor is greater than the sum of first and second cancer therapeutic component internalized and retained in a tumor if each of the first and second agents were administered alone. Also disclosed is a combination therapeutic comprising the first and second agents blended together.

This application claims the priority benefit of U.S. Provisional Patent Application Ser. No. 63/086,216, filed Oct. 1, 2020, which is hereby incorporated by reference in its entirety.

### FIELD

The present application relates to methods and compositions for synergistic uptake and retention of small molecule ligands.

### BACKGROUND

Combination therapy is a common, accepted treatment approach for virtually all types of cancers and has been the standard therapeutic approach for several decades. The basis for the adoption of combination therapy was the early chemotherapy experience where it was determined that the high mutational rate of cancers allowed rapid development of resistant strains of tumor cells when only a single agent was employed. The goal of combination therapies is to increase efficacy and minimize the development of tumor resistance or escape. This is generally achieved by employing 2 or more anti-cancer agents each of which has a different mechanism of action, making the development of resistant tumor cells more difficult and less likely. The additive or synergistic effects of combining two or more agents can be the difference between successful and unsuccessful treatment of the patient.

Many combination treatment regimens are well known in the oncology field. As an example, MOPP (an acronym for mechlorethamine, vincristine, procarbazine, prednisone) is a curative treatment regimen for Hodgkins' Disease. Several different combination regimens (which all include cisplatin, vinblastine, and bleomycin) are accepted in the treatment of testicular cancer, which is curable in up to 98% of diagnosed cases. In all, more than 300 different combination regimens have been used.

The main drawback to combination therapy is often that it also results in an increase in toxicity. For example, most forms of nonsurgical cancer therapy, such as external irradiation and chemotherapy, are limited in their efficacy because of toxic side effects to normal tissues and cells as well as the limited specificity of these treatment modalities for cancer cells.

This limitation is also of importance when anti-cancer antibodies are used for targeting toxic agents, such as isotopes, drugs, and toxins, to cancer sites, because, as systemic agents, they also circulate to sensitive cellular compartments such as the bone marrow. In acute radiation injury, there is destruction of lymphoid and hematopoietic compartments as a major factor in the development of septicemia and subsequent death. Thus, methods of reducing the toxic effects of cancer therapy while maintaining or even increasing efficacy are in high demand.

Further, although small molecules remain important drugs used in clinics, in numerous cases, their therapeutic impact has reached limitations due to insufficient capability to reach targets, lack of specificity, requirement for high doses leading to toxicity, and major side effects. Pharmaceutical potency of these molecules remains restricted by their poor stability in vivo, their rapid excretion and by their low uptake into cells. Therefore, “delivery” has become a critical piece of the therapeutic puzzle and new milestones have been established to validate delivery strategies: (a) lack of toxicity, (b) efficiency at low doses in vivo, (c) easy to handle for therapeutic applications (d) rapid endosomal release and (e) ability to reach the target. Although viral delivery strategies had given much hope for gene and cellular therapies, their clinical application has suffered from side- and toxicity-effects (Glover et al., “Towards Safe, Non-viral Therapeutic Gene Expression in Humans,” Nat. Rev. Genet. 6:299-310 (2005); Whitehead et al., “Knocking Down Barriers: Advances in siRNA Delivery,” Nat Rev Drug Discov. 8:129-138 (2009)). Researchers were mainly focused on the development of non-viral strategies, and different methods have been proposed including lipid, polycationic nanoparticles and peptide-based formulations, but only a few of these technologies have been efficient in vivo and have reached the clinic.

The present application is directed to overcoming these and other deficiencies in the art.

### SUMMARY

A first aspect of the present application relates to a method of treating a subject for cancer. The method involves providing a first agent comprising a first targeting component coupled to a first cancer therapeutic component and providing a second agent comprising a second targeting component coupled to a second cancer therapeutic component. The first and second targeting components have different biodistributions and/or pharmacokinetics in the subject. The first and second agents are administered no more than eight hours apart from each other, to the subject to treat cancer. As a result of the administering, the amount of first and second cancer therapeutic component internalized and retained within a tumor is greater than the sum of first and second cancer therapeutic component internalized and retained in a tumor if each of the first and second agents were administered individually.

A second aspect of the present application relates to a therapeutic composition that includes a first agent comprising a first targeting component coupled to a first cancer therapeutic component and a second agent, blended with the first agent. The second agent comprises a second targeting component coupled to a second cancer therapeutic component. The first and second targeting components have different biodistributions and/or pharmacokinetics in a subject.

The present application describes a way to overcome the maximum tolerated dose (MTD) of a targeted agent in order to achieve improved efficacy with no increase in, and an opportunity to decrease, its toxicity. The present application proposes the use of two individual targeting agents, rather than one, each targeting the same molecule or the same cell type. In this approach, each of the two targeted agents has a different biodistribution and/or pharmacokinetics from the other. Importantly, the different biodistributions and pharmacokinetics of these respective agents results in differing, non-overlapping toxicities of each of the two respective targeted agents. When the two targeted agents are combined in a treatment strategy, the result is that both drugs converge, simultaneously or sequentially, at the desired target site thereby providing a combined treatment effect.

Combined targeting using two different targeting agents each with different biodistributions and/pharmacokinetics is novel; it has never before been considered or utilized as described herein. Previous attempts, for example in the field of targeted radiopharmaceuticals, have been limited to use of a single targeting agent (e.g., a somatostatin receptor type 2 (SSTR-2) ligand) administered either (1) in alternating cycles carrying 2 different therapeutic moieties with a minimum of 6 weeks between cycles (Villard et al., “Cohort Study of Somatostatin-Based Radiopeptide Therapy With [⁹⁰Y-DOTA]-TOC Versus [⁹⁰Y-DOTA]-TOC Plus [¹⁷⁷Lu-DOTA]-TOC in Neuroendocrine Cancer,” J Clin Oncol 30:1100-1106 (2012), which is hereby incorporated by reference in its entirety) or (2) administering the single targeting agent with two therapeutic agents together where each therapeutic agent was given in reduced dose by 50% (Kunikowska ct al., “Clinical Results of Radionuclide Therapy of Neuroendocrine Tumors with ⁹⁰Y-DOTATATE and Tandem ⁹⁰Y/¹⁷⁷Lu-DOTATATE: Which is a Better Therapy Option?” Eur J Nucl Med Mol Imaging 38:1788-1797 (2011), which is hereby incorporated by reference in its entirety). While both of these cited publications used two radiopharmaceutical therapeutics, they were limited by the use of only one targeting agent. Since this single targeting agent would, by definition, compete with itself for binding to the target molecule, this required either alternating the respective radionuclide every other cycle or reducing the dosage of each conjugated therapeutic in order to administer simultaneously. The approach described herein solves this problem by allowing both therapeutic moieties, exemplified herein with radiopharmaceuticals, to be administered simultaneously and at full dose. This method also allows for a higher therapeutic dose to reach the target site (e.g., cancer), and therefore more efficacy. This can be achieved because the biodistribution of the two respective agents differ, they do not cause additive toxicity to normal tissues. This benefit also allows, for the first time, to overcome the prior inability to co-administer two radiopharmaceuticals where either or, especially, both are at their maximum tolerated dose/s. As another option, one may actually modulate the dose of either or both agents downward, while still delivering a greater therapeutic dose to the targeted sites than either agent individually, but also decreasing side effects by lowering the respective dose of either or both agents.

Furthermore, as demonstrated below, it has been found that not just an additive dose to tumor can be achieved, a benefit in itself, but synergy can be derived where the total dose to tumor by this approach exceeds the sum of its individual agents. This is due to the antibody altering the physiological processing of the small molecule ligand such that the latter's tumor uptake and its tumor retention are both improved and prolonged thereby delivering an even greater effective dose to tumor.

As the critical determinant of treatment efficacy is a function of the dose delivered to the intended site, the approach described offers the benefit of improved efficacy as it can deliver a higher dose to tumor without exceeding dose-limiting toxicity and patient tolerability. Furthermore, it offers the option to decrease the dose of either or both components in order to improve treatment toxicity and tolerability while still providing improved dosing relative to either agent individually.

Use of two targeting agents also allows the use of two different cytotoxic agents which provides further potential treatment benefit. For example, one may use two different radiopharmaceuticals and, in doing so, increase the curability of the treatment as described by O'Donoghue et al., “Relationships between Tumor Size and Curability for Uniformly Targeted Therapy with Beta-Emitting Radionuclides,” J Nucl Med 36:1902-1909 (1995), which is hereby incorporated by reference in its entirety. Similarly, use of two targeting agents would allow the use of two cytotoxic agents of different classes. e.g., a radiopharmaceutical and a chemotherapeutic, a pro-drug and the pro-drug's activator, a drug and it's enhancer or modulator. Multiple other combinations can be devised by those knowledgeable in the art.

The approach described herein allows enhanced specificity of therapeutic action as the only site/s in the body where the two agents would accumulate in coordination would be the targeted site/s.

### BRIEF DESCRIPTION OF THE DRAWINGS

FIG. 1 shows that, when measured via ex vivo imaging, mice treated with both 617-Lu¹⁷⁷ plus J591-Lu¹⁷⁷ have the greatest number of counts which exceeds the sum of the tumors that got either agent alone.

FIG. 2 shows that, when measured via autopsy 72 hours post-treatment, mice treated with both 617-Lu¹⁷⁷ plus J591-Lu¹⁷⁷ have the greatest number of radioactive counts which exceeds the sum of the counts in the tumors that got either agent alone.

FIG. 3 shows the combination of antibody plus small molecule ligand targeted radiopharmaceuticals delivered from 44 to 65% more radiation dose to tumor than the sum of the 2 individual agents when given individually.

### DETAILED DESCRIPTION

A first aspect of the present application relates to a method of treating a subject for cancer. The method involves providing a first agent comprising a first targeting component coupled to a first cancer therapeutic component and providing a second agent comprising a second targeting component coupled to a second cancer therapeutic component. The first and second targeting components have different biodistributions and/or pharmacokinetics in the subject. The first and second agents are administered no more than eight hours apart from each other, to the subject to treat cancer. As a result of the administering, the amount of first and second cancer therapeutic component internalized and retained within a tumor is greater than the sum of first and second cancer therapeutic component internalized and retained in a tumor if each of the first and second agents were administered individually.

As used herein, the term “subject” is intended to include human and non-human animals. Non-human animals include all vertebrates, e.g., mammals and non-mammals, such as non-human primates, sheep, dog, cow, chickens, amphibians, reptiles, etc.

As used herein, the term “treat” refers to the application or administration of the first and second agents of the application to a subject, e.g., a patient. The treatment can be to cure, heal, alleviate, relieve, alter, remedy, ameliorate, palliate, improve or affect the cancer, the symptoms of the cancer or the predisposition toward the cancer.

As used herein, the term “cancer” includes all types of cancerous growths or oncogenic processes, metastatic tissues or malignantly transformed cells, tissues, or organs, irrespective of histopathologic type or stage of invasiveness.

As used herein, the term “biodistribution” refers to the organs and tissues to which a drug distributes in the body.

As used herein, the term “pharmacokinetics” refers to how long a drug stays in the body.

In certain embodiments, the first and second agents are administered no more than 7 hours, 6 hours, 5 hours, 4 hours, 3 hours, 2 hours, or 1 hour apart from each other. In one embodiment, the first and second agents are administered simultaneously.

Regardless of the chosen time of administration of both agents, the timing should result in the amount of first and second cancer therapeutic component internalized and retained within a tumor being greater than the sum of first and second cancer therapeutic components internalized and retained in a tumor if each of the first and second agents were administered individually.

In certain embodiments, the cancer is prostate cancer, neuroendocrine cancer, breast cancer, non-Hodgkin's lymphoma, or Hodgkin's lymphoma. In some embodiments, the cancer is a primary tumor, while in other embodiments, the cancer is a secondary or metastatic tumor.

As used herein, the “targeting component” is a component that is able to bind to or otherwise associate with a molecular target, for example, a membrane component, a cell surface receptor, such as prostate specific membrane antigen (PSMA, which is also known as folate hydrolase I, glutamate carboxypeptidase II, and NAALADase), or the like. A first and second agent comprising the targeting component may become localized or converge at a particular targeted site, for instance, a tumor, a disease site, a tissue, an organ, a type of cell, etc. As such, the first and second agent may be “target-specific.” In some cases, the therapeutic component that is coupled to the first and second targeting components may exert its anti-cancer effect without the need for release from the first and second targeting components. In other cases, the therapeutic component may be released from the first and second agents and allowed to interact locally at the particular targeting site.

For example, contemplated targeting components may include a nucleic acid, peptide, polypeptide, protein, glycoprotein, carbohydrate, or lipid. A targeting component may be a naturally occurring or synthetic ligand for a cell surface receptor, e.g., a growth factor, hormone, LDL, transferrin, etc. A targeting component can be an antibody, which term is intended to include antibody fragments and/or derivatives, characteristic portions of antibodies, single chain targeting moieties which can be identified, for example, using procedures such as phage display. Targeting components may also be a targeting peptide, targeting peptidomimetic, or a small molecule, whether naturally-occurring or artificially created (e.g., via chemical synthesis).

In one embodiment, the first and second targeting components are independently selected from the group consisting of an antibody or binding fragment thereof, a protein, a peptide, and a small molecule.

Antibodies against molecular targets on tumors are known. For example, antibodies and antibody fragments which specifically bind markers produced by or associated with tumors have been disclosed, inter alia, in U.S. Pat. No. 3,927,193 to Hansen, and U.S. Pat. Nos. 4,331,647, 4,348,376, 4,361,544, 4,468,457, 4,444,744, 4,818,709 and 4,624,846 to Goldenberg, the contents of all of which are incorporated herein by reference in their entirety. In particular, antibodies against an antigen, e.g., a gastrointestinal, lung, liver, breast, prostate, kidney, bladder, ovarian, testicular, brain, hematopoietic or lymphatic tumor, a sarcoma or a melanoma, are advantageously used. Antibodies to cancer-related antigens are well known to those in the art.

The antibodies of the present application may exist in a variety of forms including, for example, polyclonal antibodies, monoclonal antibodies, intracellular antibodies (“intrabodies”), antibody fragments (e.g. Fv, Fab and F(ab)2), half-antibodies, hybrid derivatives, as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli.” Proc. Natl. Acad. Sci. USA 85:5879-5883 (1988); Bird et al, “Single-Chain Antigen-Binding Proteins,” Science 242:423-426 (1988), each of which is hereby incorporated by reference in its entirety).

Antibodies of the present application may also be synthetic antibodies. A synthetic antibody is an antibody which is generated using recombinant DNA technology, such as, for example, an antibody expressed by a bacteriophage. Alternatively, the synthetic antibody is generated by the synthesis of a DNA molecule encoding and expressing the antibody of the present application or the synthesis of an amino acid sequence specifying the antibody, where the DNA or amino acid sequence has been obtained using synthetic DNA or amino acid sequence technology which is available and well known in the art.

Methods for monoclonal antibody production may be carried out using the techniques described herein or are well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest either in vivo or in vitro.

Alternatively, monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries (McCafferty et al., “Phage Antibodies: Filamentous Phage Displaying Antibody Variable Domains,” Nature 348:552-554 (1990); Clackson et al., “Making Antibody Fragments using Phage Display Libraries.” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified using recombinant DNA technology to generate alternative antibodies. For example, the constant domains of the light and heavy chains of a human monoclonal antibody can be substituted for those regions of a mouse antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment or derivative of a monoclonal antibody. Furthermore, site-directed or high-density mutagenesis of the variable region can be used to optimize specificity and affinity of a monoclonal antibody.

The monoclonal antibody of the present application can be a humanized antibody. Humanized antibodies are antibodies that contain minimal sequences from non-human (e.g., murine) antibodies within the variable regions. Such antibodies are used therapeutically to reduce antigenicity and human anti-mouse antibody responses when administered to a human subject. In practice, humanized antibodies are typically human antibodies with minimal to no non-human sequences. A human antibody is an antibody produced by a human or an antibody having an amino acid sequence corresponding to an antibody produced by a human.

In addition to whole antibodies, the present application encompasses binding portions of such antibodies. Such binding portions include the monovalent Fab fragments, Fv fragments (e.g., single-chain antibody, scFv), and single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES:PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

It may further be desirable, especially in the case of antibody fragments, to modify the antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Antibody mimics are also suitable for use in accordance with the present application. A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J Mol. Biol. 284:1141-1151 (1998); Koide et al., “Probing Protein Conformational Changes in Living Cells by Using Designer Binding Proteins: Application to the Estrogen Receptor,” Proc. Natl. Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety).

The peptides used in conjunction with the present application can be obtained by known isolation and purification protocols from natural sources, can be synthesized by standard solid or solution phase peptide synthesis methods according to the known peptide sequence of the peptide, or can be obtained from commercially available preparations. Included herein are peptides that exhibit the biological binding properties of the native peptide and retain the specific binding characteristics of the native peptide. Derivatives and analogs of the peptide, as used herein, include modifications in the composition, identity, and derivitization of the individual amino acids of the peptide provided that the peptide retains the specific binding properties of the native peptide. Examples of such modifications would include modification of any of the amino acids to include the D-stereoisomer, substitution in the aromatic side chain of an aromatic amino acid, derivitization of the amino or carboxyl groups in the side chains of an amino acid containing such a group in a side chain, substitutions in the amino or carboxy terminus of the peptide, linkage of the peptide to a second peptide or biologically active moiety, and cyclization of the peptide (G. Van Binst and D. Tourwe. “Backbone Modifications in Somatostatin Analogues: Relation Between Conformation and Activity,” Peptide Research 5:8-13 (1992), which is hereby incorporated by reference in its entirety).

In one embodiment, the first and second targeting components target the same molecular target. For example, the first and second targeting components may bind to the same receptor (e.g. PSMA) expressed by the same cell type.

In another embodiment, the first and second targeting components target different molecular targets on the same cell type. For example, the first and second targeting components may bind to different receptors (e.g. HER1 and HER2) expressed on the same cell type.

As used herein, the “cancer therapeutic component” is an agent, or combination of agents, that treats a cell, tissue, or subject having a condition requiring therapy, when contacted with the cell, tissue or subject. The cancer therapeutic component may be, for example, a therapeutic radionuclide, chemotherapeutic agent, cytotoxin, hormone, hormone antagonist, receptor antagonist, enzyme or proenzyme activated by another agent, biologic, autocrine or cytokine. Toxins also can be used in the methods of the present application. Other therapeutic agents useful in the present application include anti-DNA, anti-RNA, radiolabeled oligonucleotides, such as anti-sense oligodeoxy ribonucleotides, anti-protein and anti-chromatin cytotoxic or antimicrobial agents. Other therapeutic agents are known to those skilled in the art, and the use of such other therapeutic agents in accordance with the present application is specifically contemplated.

In one embodiment, the first and second cancer therapeutic components are independently selected from the group consisting of a radionuclide and a cytotoxic agent.

In one embodiment, the first and/or second cancer therapeutic component is a radionuclide selected from the group consisting of ⁸⁶Re, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁸⁸Rd, ¹⁶⁶Dy, ¹⁶⁶Ho, ¹⁴⁹Pm, ¹⁵¹Pm, ¹⁵³Sm, ¹⁵⁹Gd, ¹⁷²Tm, ¹⁶⁹Yb, ¹⁷⁵Yb, ¹⁷⁷Lu, ¹⁰⁵Rh, ¹¹¹Ag, ¹³¹I. ¹⁷⁷mSn, ²²⁵Ac, ²²⁷Th, ²¹²Pb, ²¹¹At, and combinations thereof.

Procedures for labeling agents with radioactive isotopes are generally known in the art. For example, there are a wide range of moieties which can serve as chelating ligands and which can be derivatized to the targeting components of the application. For instance, the chelating ligand can be a derivative of 1,4,7,10-tetraazacyclododecanetetraacetic acid (DOTA), ethylenediaminetetraacetic acid (EDTA), diethylenetriaminepentaacetic acid (DTPA), and 1-p-Isothiocyanato-benzyl-methyl-diethylenetriaminepentaacetic acid (ITC-MX). These chelators typically have groups on the side chain by which the chelator can be used for attachment to the targeting components of the present application. Such groups include, e.g., benzylisothiocyanate, by which the DOTA, DTPA, or EDTA can be coupled to, e.g., an amine group of the targeting component. Procedures for iodinating biological agents, such as antibodies, binding portions thereof, probes, or ligands, are described by Hunter and Greenwood, “Preparation of Iodine-131 Labelled Human Growth Hormone of High Specific Activity,” Nature 144:496-496 (1962). David et al., “Protein Iodination With Solid State Lactoperoxidase,” Biochemistry 13:1014-1021 (1974), and U.S. Pat. No. 3,867,517 to Ling and U.S. Pat. No. 4,376,110 to David, which are hereby incorporated by reference in their entirety. Other procedures for iodinating biological agents are described by Greenwood et al., “The Preparation of I-131-Labelled Human Growth Hormone of High Specific Radioactivity.” Biochem. J. 89:114-123 (1963); Marchalonis, “An Enzymic Method for the Trace Iodination of Immunoglobulins and Other Proteins,” Biochem. J. 113:299-305 (1969); and Morrison et al., “Use of Lactoperoxidase Catalyzed Iodination in Immunochemical Studies,” Immunochemistry 8:289-297 (1971), which are hereby incorporated by reference in their entirety. Procedures for ^{99m}Tc-labeling are described by Rhodes, B. et al. in Burchiel, S. et al. (eds.), Tumor Imaging: The Radfoimmunochemical Detection of Cancer, New York: Masson 111-123 (1982) and the references cited therein, which are hereby incorporated by reference in their entirety. Procedures suitable for ¹¹¹In-labeling biological agents are described by Hnatowich et al., “The Preparation of DTPA-coupled Antibodies Radiolabeled With Metallic Radionuclides: an Improved Method,” J. Immul. Methods 65:147-157 (1983). Hnatowich et al., “Coupling Antibody With DTPA—an Alternative to the Cyclic Anhydride.” Int. J. Applied Radiation 35:554-557 (1984), and Buckley et al., “An Efficient Method For Labelling Antibodies With 111In,” F.E.B.S. 166:202-204 (1984), which are hereby incorporated by reference in their entirety.

In another embodiment, the cancer therapeutic component is a cytotoxic agent selected from the group consisting of busulfan, cisplatin, carboplatin, chlorambucil, cyclophosphamide, ifosfamide, dacarbazine (DTIC), mechlorethamine (nitrogen mustard), melphalan carmustine (BCNU), lomustine (CCNU), 5-fluorouracil (5-FU), capecitabine, methotrexate, gemcitabine, cytarabine (ara-C), fludarabine, dactinomycin, daunorubicin, doxorubicin (Adriamycin), idarubicin, mitoxantrone, paclitaxel, docetaxel, cabazitaxel, etoposide (VP-16), vinblastine, vincristine, vinorelbine, prednisone, dexamethasone, tamoxifen, fulvestrant, anastrozole, letrozole, megestrol acetate, bicalutamide, flutamide, leuprolide, goserelin, L-asparaginase, tretinoin, maytansines, auristatins, pyrrolobenzodiazepines, duocarmycins, and combinations thereof.

Procedures for conjugating biological agents with cytotoxic agents, such as chemotherapeutic agents, are well known in the art. Most of the chemotherapeutic agents currently in use in treating cancer possess functional groups that are amenable to chemical crosslinking directly with an amine or carboxyl group of the first targeting component of the present application. For example, free amino groups are available on methotrexate, doxorubicin, daunorubicin, cytosinarabinoside, cisplatin, vindesine, mitomycin, and bleomycin while free carboxylic acid groups are available on methotrexate, melphalan, and chlorambucil. These functional groups, that is free amino and carboxylic acids, are targets for a variety of homo-bifunctional and hetero-bifunctional chemical crosslinking agents which can crosslink these drugs directly to a free amino group of the first targeting component. Specific procedures for conjugating targeting components with chemotherapeutic agents have been described and are known in the art. By way of example, conjugation of chlorambucil with antibodies is described by Flechner, “The Cure and Concomitant Immunization of Mice Bearing Ehrlich Ascites Tumors by Treatment With an Antibody—Alkylating Agent Complex,” European Journal of Cancer 9:741-745 (1973); Ghose et al., “Immunochemotherapy of Cancer with Chlorambucil-Carrying Antibody,” British Medical Journal 3:495-499 (1972); and Szekerke et al., “The Use of Macromolecules as Carriers of Cytotoxic Groups (part II) Nitrogen Mustard—Protein Complexes,” Neoplasma 19:211-215 (1972), which are hereby incorporated by reference in their entirety. Procedures for conjugating daunomycin and adriamycin to antibodies are described by Hurwitz et al., “The Covalent Binding of Daunomycin and Adriamycin to Antibodies, With Retention of Both Drug and Antibody Activities,” Cancer Research 35:1175-1181 (1975) and Arnon et al. Cancer Survey 1:429-449 (1982), which are hereby incorporated by reference in their entirety. Coupling procedures are also described in EP 86309516.2, which is hereby incorporated by reference in its entirety.

It will be appreciated that the exact dosage of the first and second agents of the application is chosen by the individual physician in view of the patient to be treated. In general, dosage and administration are adjusted to provide an effective amount of the agent to the patient being treated. As used herein, the “effective amount” of an agent refers to the amount necessary to elicit the desired biological response. As will be appreciated by those of ordinary skill in this art, the effective amount of agent may vary depending on such factors as the desired biological endpoint, the drug to be delivered, the target tissue, the route of administration, etc. For example, the effective amount of agent containing an anti-cancer drug might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

In general, doses can range from about 25% to about 100% of the MTD of the targeted agent when given as a single agent. Based upon the composition, the dose can be delivered once, continuously, such as by continuous pump, or at periodic intervals. Dosage may be adjusted appropriately to achieve desired drug levels, locally, or systemically. In the event that the response in a subject is insufficient at such doses, even higher doses (or effective higher doses by a different, more localized delivery route) may be employed to the extent that patient tolerance permits. Continuous IV dosing over, for example, 24 hours or multiple doses per day also are contemplated to achieve appropriate systemic levels of compounds.

In one embodiment, the cancer therapeutic component has a maximum tolerated dose, and the maximum tolerated dose of the cancer therapeutic component is administered to the subject. Because the biodistribution and pharmacokinetics are different for the two targeting components, their toxicities as individual drugs are non- or minimally overlapping. As a result, the increased, additive dose to the target site is not accompanied by a commensurate increase in toxicity.

In an alternative embodiment, less than the maximum tolerated dose of the cancer therapeutic component is administered to the subject. When the two agents of the present application are combined in a treatment strategy, the result is that both agents converge (simultaneously or sequentially) at the desired target site thereby providing a synergistic treatment effect and, because the therapeutic component of the first and second agent is administered at less than its MTD, lower toxicity is experienced by the subject.

In one embodiment, the first and/or second agent is a small molecule conjugated to a radionuclide Lu¹⁷⁷ and is administered in a 2-week cycle at a total dose of about 300 to 900 mCi (11.0-33.3 GBq), such as a dose of 300, 325, 350, 375, 400, 425, 450, 475, 500, 600, 700, 800 or 900 mCi total in a 2 week cycle. In another embodiment, the small molecule conjugated to radionuclide Lu¹⁷⁷ is administered at 6-week intervals or between 4 to 8-week cycles or longer. In this case, each dose ranges from 5.0-9.0 GBq (135-243mCi).

In practicing the methods of the present application, the administering step is carried out to treat cancer in a subject. In one embodiment, a subject having cancer is selected prior to the administering step. Such administration can be carried out systemically or via direct or local administration to the tumor site. By way of example, suitable modes of systemic administration include, without limitation, orally, topically, transdermally, parenterally, intradermally, intramuscularly, intraperitoneally, intravenously, subcutaneously, or by intranasal instillation, by intracavitary or intravesical instillation, intraocularly, intraarterialy, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent (e.g., an antibody or an inhibitory nucleic acid molecule) and the disease to be treated.

The agents of the present application may be orally administered, for example, with an inert diluent, or with an assimilable edible carrier, or it may be enclosed in hard or soft shell capsules, or it may be compressed into tablets, or they may be incorporated directly with the food of the diet. Agents of the present application may also be administered in a time release manner incorporated within such devices as time-release capsules or nanotubes. Such devices afford flexibility relative to time and dosage. For oral therapeutic administration, the agents of the present application may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present application in such therapeutically useful compositions is such that a suitable dosage will be obtained.

When the agents of the present application are administered parenterally, solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present application systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present application can also be achieved using infusion pump devices. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt

According to one embodiment, the cancer is prostate cancer.

In another embodiment of this aspect of the present application, when the cancer is prostate cancer, the first and second targeting components target the PSMA receptor.

As used herein, “PSMA” or “prostate-specific membrane antigen” protein refers to mammalian PSMA, preferably human PSMA protein. PSMA is also known as Folate Hydrolase 1. The long transcript of PSMA encodes a protein product of about 100-120 kDa molecular weight characterized as a type II transmembrane receptor having sequence homology with the transferrin receptor and having NAALADase activity (Carter et al., “Prostate-Specific Membrane Antigen is a Hydrolase With Substrate and Pharmacologic Characteristics of a Neuropeptidase,” Proc. Natl. Acad Sci. USA 93:749-753 (1996), which is hereby incorporated by reference in its entirety).

In an alternative embodiment, the first targeting component is a PSMA receptor antibody or antigen binding portion thereof and the second targeting component is a PSMA receptor binding peptide or PSMA receptor inhibitor.

A PSMA receptor antibody is an antibody that interacts with (e.g., binds to) PSMA, preferably human PSMA protein. Preferably, the PSMA receptor antibody interacts with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866, which is hereby incorporated by reference in its entirety). PSMA receptor antibodies are known in the art (Goldsmith et al., “Targeted Radionuclide Therapy for Prostate Cancer.” in Therapeutic Nuclear Medicine 617-628 (R. Baum ed. 2014), which is hereby incorporated by reference in its entirety). Exemplary PSMA receptor antibodies include, but are not limited to, J591, J415, J533, and E99 (Tykvart et al., “Comparative Analysis of Monoclonal Antibodies Against Prostate-specific Membrane Antigen (PSMA).” The Prostate 74(16):1674-90 (2014), which is hereby incorporated by reference in its entirety).

The PSMA receptor inhibitor may include any lipids, carbohydrates, polynucleotides, peptides, polypeptides, or any other biologic, organic or inorganic molecules which inhibit the function of the PSMA receptor. Exemplary PSMA receptor inhibitor are known in the art include, but are not limited to, PSMA 617, PSMA I&T, DCFBC, DCFPyL, glutamate-urea-lysine analogs, phosphoramidate analogs, and 2-(phosphinylmethyl) pentanedioic acid analogs (Lutje et al., “PSMA Ligands for Radionuclide Imaging and Therapy of Prostate Cancer: Clinical Status,” Theranostics 5(12):1388-1401 (2015); Haberkorn et al., “New Strategies in Prostate Cancer: Prostate-Specific Membrane Antigen (PSMA) Ligands for Diagnosis and Therapy,” Clin. Cancer Res. 22(1):9-15 (2016), which are hereby incorporated by reference in their entirety).

In one embodiment, the first targeting component is an antibody selected from the group consisting of J591, J415, J533, and E99, while the second targeting component is a peptide selected from the group consisting of PSMA 617, PSMA I&T, DCFBC, DCFPyL, glutamate-urea-lysine analogs, phosphoramidate analogs, 2-(phosphinylmethyl) pentanedioic acid analogs, and other PSMA ligands/inhibitors/peptides.

In one embodiment, the first agent is J591-¹⁷⁷Lu or J591-²²⁵Ac and the second agent is PSMA 617-¹⁷⁷Lu, PSMA 617-²²³Ac, PSMA I&T-¹⁷⁷Lu, or PSMA I&T-²²⁵Ac. Either agent may carry an alpha or a beta emitting radionuclide resulting in a variety of pairs such as: alpha/alpha, beta/beta, alpha/beta or beta/alpha. In another embodiment, the targeting agents may carry other classes of cytotoxic agents (e.g., drug or toxin) well known to those in the art.

In another embodiment of the present application, the cancer is a neuroendocrine cancer. Neuroendocrine cancers include, but are not limited to, carcinoid tumors, gastrinoma, insulinoma, glucagonoma, VIPoma, somatostatinoma, thyroid carcinoma, Merkel cell carcinoma of the skin, tumor of the anterior pituitary, medullary carcinoma, parathyroid tumor, thymus and mediastinal carcinoid tumor, pulmonary neuroendocrine tumor, adrenomedullary tumor, pheochromocytoma, Schwannoma, paraganglioma, neuroblastoma, and urinary tract carcinoid neumendocrine carcinoma.

In accordance with this aspect of the present application, in one embodiment, the first and second targeting components target the somatostatin receptor.

At least five somatostatin receptors subtypes have been characterized and tumors can express various receptor subtypes (Shaer et al., “Somatostatin Receptor Subtypes sst1, sst2, sst3 and sst5 Expression in Human Pituitary, Gastroentero-Pancreatic and Mammary tumors: Comparison of mRNA Analysis With Receptor Autoradiography,” Int. J. Cancer 70:530-537 (1997), which is hereby incorporated by reference in its entirety). Naturally occurring somatostatin and its analogs exhibit differential binding to these receptor subtypes, allowing precise targeting of a peptide analog to specific diseased tissues.

In accordance with this aspect of the application, the first and second targeting components have at least one biological activity of native somatostatin; preferably, this activity is the ability to specifically bind to a somatostatin receptor on a somatostatin receptor-bearing cell. Many such analogs having biological activity are known and have been described, for example, in U.S. Pat. No. 5,770,687 to Homik et al.; U.S. Pat. No. 5,708,135 to Coy et al.; U.S. Pat. No. 5,750,499 to Hoeger et al; U.S. Pat. No. 5,620,675 to McBride et al.; U.S. Pat. No. 5,633,263 to Coy et al; U.S. Pat. No. 5,597,894 to Coy et al; U.S. Pat. No. 5,073,541 to Taylor et al; U.S. Pat. No. 4,904,642 to Coy et al; U.S. Pat. No. 6,017,509 to Dean; WO 98/47524 to Hoffman et al.; and U.S. Pat. No. 5,411,943 to Bogden, each of which is hereby incorporated by reference in its entirety.

In one embodiment, the first and second targeting components target the somatostatin receptor-2.

In another embodiment of the present application the cancer is breast cancer.

In accordance with this embodiment of the present application, when the cancer is breast cancer, the first and second targeting components target the HER receptor family.

First and second agents, as well as targeting and therapeutic components, are described above.

In another embodiment of the present application the cancer is non-Hodgkin's Lymphoma.

In accordance with this embodiment, when the cancer is non-Hodgkin's lymphoma, the first and second targeting components target CD20.

In another embodiment of the present application the cancer is Hodgkin's disease.

In accordance with this embodiment, when the cancer is Hodgkin's disease, the first and second targeting components target CD30.

Another aspect of the present application relates to a therapeutic composition that includes a first agent comprising a first targeting component coupled to a first cancer therapeutic component and a second agent, blended with the first agent. The second agent comprises a second targeting component coupled to a second cancer therapeutic component. The first and second targeting components have different biodistributions and/or pharmacokinetics in a subject.

First and second agents, as well as targeting and therapeutic components, are described above.

Pharmaceutical compositions containing agents for use in the methods of the present application can include a pharmaceutically acceptable carrier as described infra, one or more active agents, and a suitable delivery vehicle. Suitable delivery vehicles include, but are not limited to, viruses, bacteria, biodegradable microspheres, microparticles, nanoparticles, liposomes, collagen minipellets, and cochleates.

In one embodiment of the present application, the pharmaceutical composition or formulation containing an inhibitory nucleic acid molecule (e.g., siRNA molecule) is encapsulated in a lipid formulation to form a nucleic acid-lipid particle as described in Semple et al., “Rational Design of Cationic Lipids for siRNA Delivery,” Nature Biotech. 28:172-176 (2010), WO2011/034798 to Bumcrot et al., WO2009/111658 to Bumcrot et al., and WO2010/105209 to Bumcrot et al., which are hereby incorporated by reference in their entirety.

In another embodiment of the present application, the delivery vehicle is a nanoparticle. A variety of nanoparticle delivery vehicles are known in the art and are suitable for delivery of an inhibitor of the application (see e.g., van Vlerken et al., “Multi-functional Polymeric Nanoparticles for Tumour-Targeted Drug Delivery,” Expert Opin. Drug Deliv. 3(2):205-216 (2006), which is hereby incorporated by reference in its entirety). Suitable nanoparticles include, without limitation, poly(beta-amino esters) (Sawicki et al., “Nanoparticle Delivery of Suicide DNA for Epithelial Ovarian Cancer Cell Therapy.” Adv. Exp. Med. Biol. 622:209-219 (2008), which is hereby incorporated by reference in its entirety), polyethylenimine-alt-poly(ethylene glycol) copolymers (Park et al., “Degradable Polyethylenimine-alt-Poly(ethylene glycol) Copolymers As Novel Gene Carriers,” J. Control Release 105(3):367-80 (2005) and Park et al., “Intratumoral Administration of Anti-KITENIN shRNA-Loaded PEI-alt-PEG Nanoparticles Suppressed Colon Carcinoma Established Subcutaneously in Mice,” J Nanosci. Nanotechnologv 10(5):3280-3 (2010), which are hereby incorporated by reference in their entirety), and liposome-entrapped siRNA nanoparticles (Kenny et al., “Novel Multifunctional Nanoparticle Mediates siRNA Tumor Delivery, Visualization and Therapeutic Tumor Reduction In Vivo,” J. Control Release 149(2): 111-116 (2011), which is hereby incorporated by reference in its entirety). Other nanoparticle delivery vehicles suitable for use in the present application include microcapsule nanotube devices disclosed in U.S. Patent Publication No. 2010/0215724 to Prakash et al., which is hereby incorporated by reference in its entirety.

In another embodiment of the present application, the pharmaceutical composition is contained in a liposome delivery vehicle. The term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Several advantages of liposomes include: their biocompatibility and biodegradability, incorporation of a wide range of water and lipid soluble drugs; and they afford protection to encapsulated drugs from metabolism and degradation. Important considerations in the preparation of liposome formulations are the lipid surface charge, vesicle size, and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Methods for preparing liposomes for use in the present application include those disclosed in Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda, and U.S. Pat. No. 5,059,421 to Loughrey et al., which are hereby incorporated by reference in their entirety.

In another embodiment of the present application, the delivery vehicle is a viral vector. Viral vectors are particularly suitable for the delivery of inhibitory nucleic acid molecules, such as siRNA or shRNA molecules, but can also be used to deliver molecules encoding an anti-integrin antibody. Suitable gene therapy vectors include, without limitation, adenoviral vectors, adeno-associated viral vectors, retroviral vectors, lentiviral vectors, and herpes viral vectors.

Adenoviral viral vector delivery vehicles can be readily prepared and utilized as described in Berkner, “Development of Adenovirus Vectors for the Expression of Heterologous Genes,” Biotechniques 6:616-627 (1988). Rosenfeld et al., “Adenovirus-Mediated Transfer of a Recombinant Alpha 1-Antitrypsin Gene to the Lung Epithelium In Vivo,” Science 252:431-434 (1991), WO 93/07283 to Curiel et al., WO 93/06223 to Perricaudet et al., and WO 93/07282 to Curiel et al., which are hereby incorporated by reference in their entirety. Adeno-associated viral delivery vehicles can be constructed and used to deliver an inhibitory nucleic acid molecule of the present application to cells as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-Aβ Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); and Zhou et al., “Adeno-associated Virus 2-Mediated Transduction and Erythroid Cell-Specific Expression of a Human Beta-Globin Gene,” Gene Ther. 3:223-229 (1996), which are hereby incorporated by reference in their entirety. In vivo use of these vehicles is described in Flotte et al., “Stable in Vivo Expression of the Cystic Fibrosis Transmembrane Conductance Regulator With an Adeno-Associated Virus Vector,” Proc. Nat'l. Acad Sci. 90:10613-10617 (1993) and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Retroviral vectors which have been modified to form infective transformation systems can also be used to deliver a nucleic acid molecule to a target cell. One such type of retroviral vector is disclosed in U.S. Pat. No. 5,849,586 to Kriegler et al., which is hereby incorporated by reference. Other nucleic acid delivery vehicles suitable for use in the present application include those disclosed in U.S. Patent Publication No. 20070219118 to Lu et al., which is hereby incorporated by reference in its entirety.

Regardless of the type of infective transformation system employed, it should be targeted for delivery of the nucleic acid to the desired cell type. For example, for delivery into a cluster of cells (e.g., cancer cells) a high titer of the infective transformation system can be injected directly within the site of those cells so as to enhance the likelihood of cell infection. The infected cells will then express the inhibitory nucleic acid molecule targeting the inhibition of integrin expression. The expression system can further contain a promoter to control or regulate the strength and specificity of expression of the nucleic acid molecule in the target tissue or cell.

Effective doses of the compositions of the present application vary depending upon many different factors, including type and stage of cancer, means of administration, target site, physiological state of the patient, other medications or therapies administered, and physical state of the patient relative to other medical complications. Treatment dosages need to be titrated to optimize safety and efficacy.

### EXAMPLES

The following examples are provided to illustrate embodiments of the present application, but they are by no means intended to limit its scope.

### Example 1—Mice Treated with Both 617-Lu¹⁷⁷ Plus J591-Lu¹⁷⁷ have the Greatest Number of Counts Measured Via Ex Vivo Imaging which Exceeds the Sum of the Tumors that Got Either Agent Alone

In this animal study, three groups of mice were implanted with LNCaP tumors (in Matrigel) with 6-8 tumors per group. After the tumors were established, one group was injected with J591-Lu¹⁷⁷, a 2^{nd} group with PSMA-617-Lu¹⁷⁷ and a 3^{rd} group got both PSMA617-Lu¹⁷⁷ and J591-Lu¹⁷⁷.

| | | |
|---|---|---|
| 1. | J591-Lu¹⁷⁷ | 150 μCi/200 μL per mouse |
| 2. | PSMA-617-Lu¹⁷⁷ | 400 μCi/200 μL per mouse |
| 3. | J591-Lu¹⁷⁷ + PSMA-617-Lu¹⁷⁷ | 150 μCi + 400 μCi/200 μL per mouse |

Radiolabeling both PSMA-617 and J591 allowed both to be tracked individually as well as their total when combined. All mice were imaged in a Siemens Inveon dedicated animal multimodality SPECT/CT. Coaxial CT images were acquired for anatomical and attenuation information prior to each SPECT acquisition to measure the radioactivity within the tumors at the 1, 4, 24 and 72 hour time points after injection. SPECT images were reconstructed using the Siemens OSEM algorithm with corrections for scatter, attenuation, decay, and ¹⁷⁷Lu emission spectrum. The mean number of counts within the tumors in each group and time point was calculated and plotted. THe imaging data measured an activity concentration in Bq/mL of tumor:

| TREATMENT GROUP | 1 hr | 4 hr | 24 hr | 72 hr |
|---|---|---|---|---|
| J591 ONLY | 1.03167 | 2.19515 | 14.05634 | 49.57959 |
| PSMA 617 ONLY | 29.54551 | 30.62378 | 27.09825 | 12.48065 |
| Summation of PSMA 617 + J591 | 30.57719 | 32.81893 | 41.15459 | 62.06024 |
| PSMA 617 + J591 (measured) | 58.10495 | 77.34706 | 84.46413 | 94.65056 |

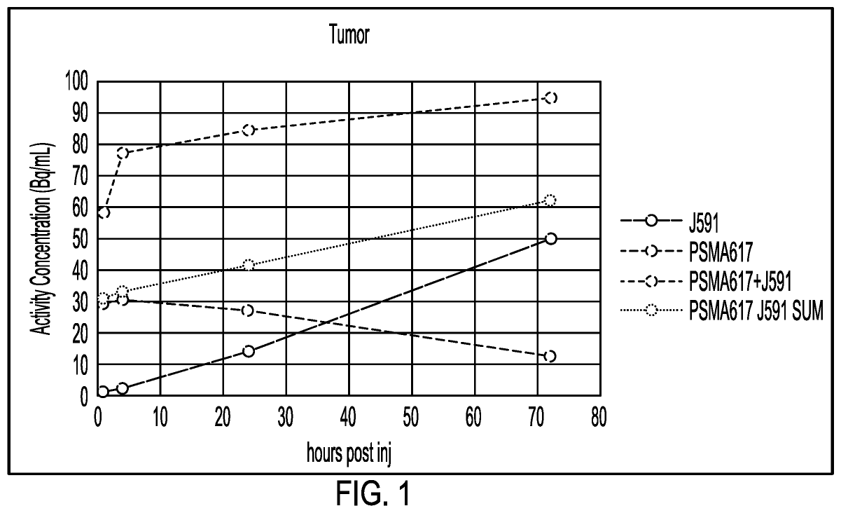

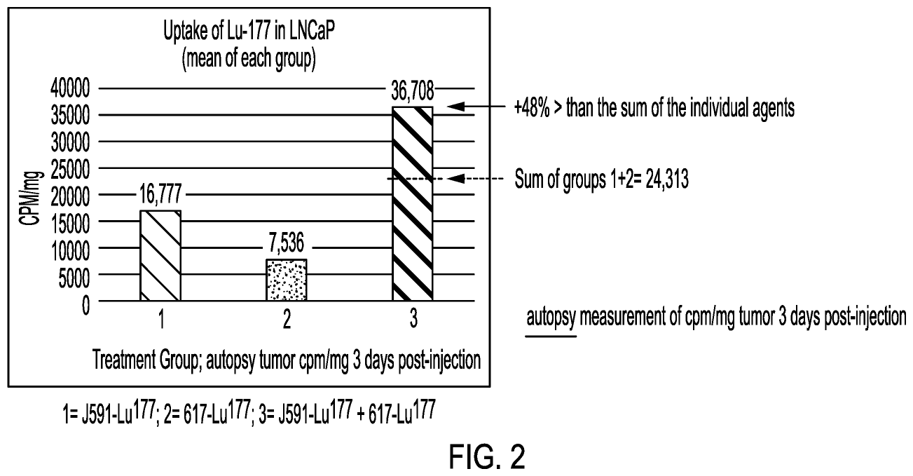

The mice that got PSMA-617-Lu¹⁷⁷ alone had a high uptake at 1 hour that then diminished over time—as plotted in FIG. 1. Conversely, those tumors treated with J591-Lu¹⁷⁷ had relatively low counts initially but a continuous increase over time for the full 72 hour timeframe. The dotted line represents the summation of the counts in the 617-Lu¹⁷⁷ plus the J591-Lu¹⁷⁷ tumors. The 3^{rd} group of animals that were treated with both 617-Lu¹⁷⁷ plus J591-Lu¹⁷⁷ had the greatest number of counts which, in fact, exceeded the sum of the tumors that got either agent alone, meeting the definition of synergy that is greater than the sum of the parts (FIG. 2). To confirm the accuracy of this experiment which was based on ex vivo imaging, determinations were done by tumor harvest at necropsy followed by direct counting of the radioactivity within the previously imaged tumors and calculating counts per mg of tumor.

At the Day 3 tumor harvest, the mean counts per mg tumor in each of the groups is shown in the table below (and FIG. 2) further confirming the imaging results.

| | | PSMA- | J591-Lu¹⁷⁷ + |
|---|---|---|---|
| Treatment: | J591-Lu¹⁷⁷ | 617-Lu¹⁷⁷ | PSMA-617-Lu¹⁷⁷ |
| Mean cpm/mg tumor | 16.777 | 7.536 | 36.708 |

### Example 2—Mice Treated with Both 617-Lu¹⁷⁷ Plus J591-Lu¹⁷⁷ have the Greatest Number of Counts Exceeding the Sum of the Tumors that Got Either Agent Alone

In this animal study, which is a continuation of the imaging study described in Example 1, when the imaging was completed at 72 hours, the animals were necropsied, the tumors harvested, weighed and counted in a scintillation counter to derive the amount of radioactivity per mg (cpm/mg) of tumor. The tumors treated with J591-Lu¹⁷⁷ had 2.2-fold the counts of those tumors that had gotten PSMA-617-Lu¹⁷⁷ alone (FIG. 2). The tumors treated with the combination had counts 48% greater than the sum of each of its individual components, meeting the definition of synergy (FIG. 2). The physical counting of the tumor radioactivity confirmed the results obtained by imaging.

### Example 3—the Combination of Antibody Plus Small Molecule Ligand Targeted Radiopharmaceuticals Delivered from 44 to 65% More Radiation Dose to Tumor than the Sum of the 2 Individual Agents when Given Individually

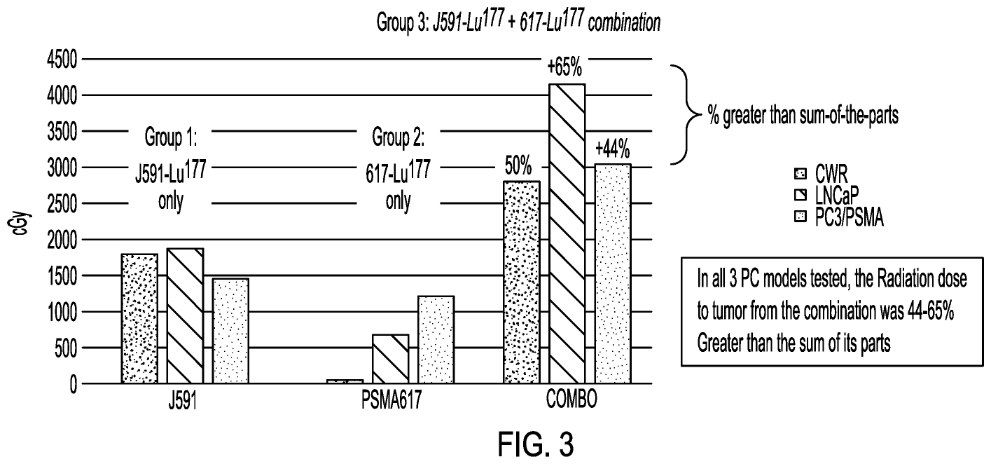

A series of experiments were done to determine the radiation dosimetry delivered to 3 different human prostate cancer models (LNCaP, CWR22Rv1q and PC3-PSMA-pos (by transfection). 3 different treatment regimens were used: 617-Lu¹⁷⁷ alone, J591-Lu¹⁷⁷ alone and the combination. As in all the experiments described here, each animal got one dose. Dosimetry calculations were based on sequential tumor imaging (as described above in Example 1). Cumulated activity was calculated and absorbed dose estimated using ¹⁷⁷Lu dose information from the Medical Internal Radiation Dose (MIRD): Radionuclide Data and Decay Schemes. The findings demonstrate that, in the 3 xenograft models, the combination of antibody plus small molecule ligand targeted radiopharmaceuticals delivered from 44 to 65% more radiation dose to tumor that the sum of the 2 individual agents when given individually (FIG. 3).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating a subject for cancer, said method comprising:
providing a first agent comprising a first targeting component coupled to a first cancer therapeutic component;
providing a second agent comprising a second targeting component coupled to a second cancer therapeutic component, wherein the first and second targeting components have different biodistributions and/or pharmacokinetics in the subject; and
administering the first and second agents, no more than eight hours apart from each other, to the subject to treat cancer, wherein, as a result of said administering, the amount of first and second cancer therapeutic component internalized and retained within a tumor is greater than the sum of first and second cancer therapeutic components internalized and retained in a tumor if each of the first and second agents were administered individually.

2. The method according to claim 1, wherein the first and second agents are administered no more than 6 hours apart from each other.

3. The method according to claim 1, wherein the first and second agents are administered no more than 4 hours apart from each other.

4. The method according to claim 1, wherein the first and second agents are administered no more than 2 hours apart from each other.

5. The method according to claim 1, wherein the first and second agents are administered simultaneously.

6. The method according to claim 1, wherein the first and second targeting components are independently selected from the group consisting of an antibody or antigen binding fragment or derivative thereof, a protein, a peptide, and a small molecule.

7. The method according to claim 1, wherein the first and second targeting components target the same molecular target.

8. The method according to claim 1, wherein the first and second targeting components target different molecular targets on the same cell.

9. The method according to claim 1, wherein the first and second cancer therapeutic components each have a maximum tolerated dose, and the maximum tolerated doses of the first and second cancer therapeutic components are given during said administering.

10. The method according to claim 1, wherein the first and second cancer therapeutic components each have a maximum tolerated dose, and less than the maximum tolerated doses of the first and second cancer therapeutic components are given during said administering.

11. The method according to claim 1, wherein the first and second cancer therapeutic components are independently selected from the group consisting of a radionuclide and a cytotoxic agent.

12. The method according to claim 11, wherein the first and/or second cancer therapeutic component is a radionuclide independently selected from the group consisting of ⁸⁶Re, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁸⁸Rd, ¹⁶⁶Dy, ¹⁶⁶Ho, ¹⁴⁹Pm, ¹⁵¹Pm, ¹⁵³Sm, ¹⁵⁹Gd, ¹⁷²Tm, ¹⁶⁹Yb, ¹⁷⁵Yb, ¹⁷⁷Lu, ¹⁰⁵Rh, ¹¹¹Ag, ¹³¹I, ^{177m}Sn, ²²⁵Ac, ²²⁷Th, ²¹²Pb, ²¹¹At, and combinations thereof.

13. The method according to claim 11, wherein the first and/or second cancer therapeutic component is a cytotoxic agent independently selected from the group consisting of busulfan, cisplatin, carboplatin, chlorambucil, cyclophosphamide, ifosfamide, dacarbazine (DTIC), mechlorethamine (nitrogen mustard), melphalan carmustine (BCNU), lomustine (CCNU), 5-fluorouracil (5-FU), capecitabine, methotrexate, gemcitabine, cytarabine (ara-C), fludarabine, dactinomycin, daunorubicin, doxorubicin (Adriamycin), idarubicin, mitoxantrone, paclitaxel, docetaxel, cabazitaxel, etoposide (VP-16), vinblastine, vincristine, vinorelbine, prednisone, dexamethasone, tamoxifen, fulvestrant, anastrozole, letrozole, megestrol acetate, bicalutamide, flutamide, leuprolide, goserelin, L-asparaginase, tretinoin, maytansines, auristatins, pyrrolobenzodiazepines, duocarmycins, and combinations thereof.

14. The method according to claim 1, wherein the cancer is prostate cancer.

15. The method according to claim 14, wherein the first and second targeting components target the prostate-specific membrane antigen (PSMA) receptor.

16. The method according to claim 15, wherein the first targeting component is a PSMA receptor antibody or antigen binding portion thereof and the second targeting component is a PSMA receptor binding peptide or PSMA receptor inhibitor.

17. The method according to claim 16, wherein the first targeting component is an antibody selected from the group consisting of J591, J415, J533, and E99, while the second targeting component is a peptide selected from the group consisting of PSMA 617, PSMA I&T, DCFBC, DCFPyL, glutamate-urea-lysine analogs, phosphoramidate analogs, 2-(phosphinylmethyl) pentanedioic acid analogs, and other PSMA ligands/inhibitors/peptides.

18. The method according to claim 16, wherein the first agent is J591-¹⁷⁷Lu or J591-²²⁵Ac and the second agent is PSMA 617-¹⁷⁷Lu, PSMA I&T-¹⁷⁷Lu, PSMA 617-²²⁵Ac, or PSMA I&T-²²⁵Ac.

19. The method according to claim 1, wherein the subject is a human.

20. The method according to claim 1, wherein the cancer is a neuroendocrine cancer, breast cancer, non-Hodgkin's Lymphoma, or Hodgkin's disease.

21-27. (canceled)

28. The method according to claim 1, wherein the first and second agents are different.

29. The method according to claim 1, wherein the first and second targeting components target a cancer cell membrane molecule.

30. A therapeutic composition comprising:
a first agent comprising a first targeting component coupled to a first cancer therapeutic component and
a second agent, blended with said first agent, said second agent comprising a second targeting component coupled to a second cancer therapeutic component, wherein the first and second targeting components have different biodistributions and/or pharmacokinetics in a subject.

31-38. (canceled)
